(19) United States
(12) Patent Application Publication
(10) Pub. No.: US 2026/0315086 A1
(43) Pub. Date: Oct. 15, 2026

(54) CATHETER DEVICES, ATHERECTOMY SYSTEMS, AND METHODS FOR REMOVING OCCLUSIVE MATERIAL FROM A TARGET SITE

(71) Applicant: BARD PERIPHERAL VASCULAR, INC., Franklin Lakes, NJ (US)

(72) Inventors: Alexander HENDRICKSON, Goodyear, AZ (US); Austin JONES, Fuquay-Varina, NC (US); Aseem SINGH, Tempe, AZ (US)

(73) Assignee: BARD PERIPHERAL VASCULAR, INC., Franklin Lakes, NJ (US)

(21) Appl. No.: 19/484,778

(22) PCT Filed: May 25, 2023
(86) PCT No.: PCT/US2023/023614

§ 371 Date: Nov. 14, 2025

(51) Int. Cl. A61B 17/22; A61B 17/3207; A61F 2/01

(52) U.S. Cl. CPC A61B 17/2202; A61B 17/320725; A61F 2/011; A61F 2/013

(57) ABSTRACT

A catheter device includes a body extending between a proximal portion and a distal portion and defining a lumen and at least one lateral opening in communication with the lumen at the distal portion. At least one vibrational pad is positioned within the lumen to adjustably protrude radially through the at least one lateral opening, such that the at least one vibrational pad is energized at a frequency and amplitude sufficient to break apart sub-intimal or medial plaque.

### TECHNICAL FIELD

The present disclosure relates to catheter devices, and, more particularly, to a vibrational catheter for removing occlusive material from a target site.

### BACKGROUND

Catheter devices may be used to aid in removing occlusive material (e.g., plaque) located in a luminal space using various methods of ablation (e.g., thermal, mechanical, cutting, laser, etc.). However, such devices are ineffective for removal of both luminal and medial occlusive material. Accordingly, a need exists for catheters, atherectomy systems, and methods for removing occlusive material located both luminally and/or subintimally/medially.

### SUMMARY

Embodiments of the present disclosure provide catheters, atherectomy systems, and methods for removing occlusive material located both luminally and/or subintimally/medially. For example, in some embodiments, a catheter device according to the present disclosure may utilize vibrational energy to disintegrate plaque at a target site in a vessel wall.

In one embodiment, a catheter device includes a body extending between a proximal portion and a distal portion and defining a lumen and at least one lateral opening in communication with the lumen at the distal portion. The catheter device further includes at least one vibrational pad positionable within the lumen and configured to adjustably protrude radially through the at least one lateral opening, such that the at least one vibrational pad is configured to be energized at a frequency and amplitude sufficient to break apart plaque.

In another embodiment, an atherectomy system includes a catheter having a body extending between a proximal portion and a distal portion and defining a lumen and at least one lateral opening in communication with the lumen at the distal portion. The catheter further includes at least one vibrational pad positionable within the lumen and configured to adjustably protrude radially through the at least one lateral opening, such that the one vibrational pad is configured to be energized at a frequency and amplitude sufficient to break apart plaque. The atherectomy system further includes a filter configured to be positioned distally to the distal portion of the catheter, the filter having a retracted position and an expanded position in which the filter is configured to extend across the lumen.

In yet another embodiment, a method of removing plaque from a target site is disclosed. The method may include introducing a distal end of a catheter having at least one vibrational pad to the target site, and protruding the at least one vibrational pad from the distal end of the catheter such that the at least one vibrational pad contacts plaque located at the target site. The method further includes energizing the at least one vibrational pad such that vibration of the at least one vibrational pad dislodges plaque located at the target site, and capturing, using a filter device positioned in the distal end of the catheter, the plaque dislodged from the target site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a longitudinal side view of a catheter device including at least one vibration pad, according to one or more embodiments described herein;

FIG. 2 schematically depicts a longitudinal side view of the catheter device of FIG. 1 positioned within a vessel with the at least one vibrational pad in a first position, according to one or more embodiments described herein;

FIG. 3 schematically depicts a longitudinal side view of the catheter device of FIG. 2 with the at least one vibrational pad in a second position, according to one or more embodiments described herein;

FIG. 4A schematically depicts a longitudinal side view of a catheter device having a plurality of vibrational pads in a first position, according to one or more embodiments described herein;

FIG. 4B schematically depicts a longitudinal side view of the catheter device of FIG. 4A with the plurality of vibrational pads in a second position, according to one or more embodiments described herein;

FIG. 4C schematically depicts a longitudinal side view of the catheter device of FIG. 4B disposed within a vessel, according to one or more embodiments described herein;

FIG. 5 schematically depicts a diagram of a controller for operating a catheter device, according to one or more embodiments described herein;

FIG. 6A schematically depicts a longitudinal side view of an atherectomy system positioned within a vessel and having a filter in a retracted position and including the catheter device, according to one or more embodiments described herein;

FIG. 6B schematically depicts a longitudinal side view of the atherectomy system of FIG. 6A having the filter in an expanded position, according to one or more embodiments described herein; and

FIG. 7 depicts a flow diagram depicting a method of removing occlusive material from a target site using an atherectomy system, according to one or more embodiments described herein.

Corresponding reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to catheter devices, atherectomy systems and methods of removing occlusive material (e.g., plaque thrombi, clots, etc.) from a target site. For example, in embodiments, a catheter device may include a body having at least one vibrational pad positioned on a distal end of the body. The vibrational pad may be actuated between a first position, in which the vibrational pad is substantially flush with an external surface of the body, and a second position, in which the vibrational pad protrudes from an external surface of the body in a radial direction. In the second position, the vibrational pad may protrude from the external surface of the body such that the vibrational pad contacts plaque located at a target site. The at least one vibrational pad may further be compliant such that the at least one vibrational pad may conform to the target site on an arterial wall on which the plaque is located. The at least one vibrational pad may be energized at a frequency and amplitude sufficient to break apart, for example, occlusive material. In some embodiments, vibration of the at least one vibrational pad may alternate between transverse vibration and longitudinal vibration in order to increase the efficacy of occlusive material disintegration. By applying transverse and longitudinal vibrational energy to the occlusive materials positioned at the target site, the catheter device may more effectively disintegrate the occlusive materials thereby removing blockages, and/or reversing narrowing of the vessel. These and additional features will be described in greater detail below.

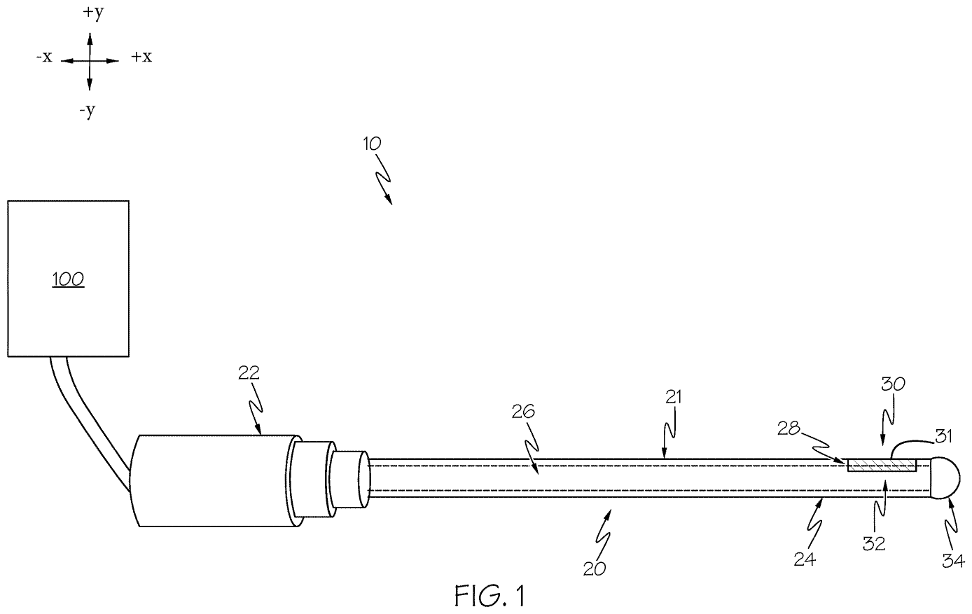

Referring now to FIG. 1, a catheter device 10 is schematically depicted. The catheter device generally includes a body 20 extending between a proximal end 22 and a distal end 24. The body 20 may define a lumen 26, and at least one lateral opening 28 may be positioned on the distal end 24 of the body 20 in communication with the lumen 26. The distal end 24 of the body 20 of the catheter device 10 may further include a tip 34, such as a silicon tip, which may be coupled to the body 20 (e.g., welded, adhered, etc.). In some embodiments, it is contemplated that the tip 34 is integral with the body 20. The tip 34 of the catheter device 10 may be shaped, e.g., atramatically, to allow the catheter device 10 to traverse a lumen of a vessel (e.g., a vein or an vessel) to a target location such as an area having occlusive material (e.g., plaque) which may be causing narrowing or occluding the vessel. For example the tip 34 of the catheter device 10 may include a cone, dome, or other similar shape that may aid the catheter device 10 in traversing tortuous and stenosed vessels. Furthermore, in other embodiments, the tip 34 of the catheter device 10 may include a vibration mechanism or otherwise be vibrated such as via an ultrasound transducer, that may allow for the catheter device 10 to traverse difficult such as dense or hard lesions and ensure that the catheter device 10 does not become stuck within a vessel. It should be appreciated that, in these embodiments, the occlusive material may include luminal, medial, and/or sub-intimal plaque (e.g., plaque embedded within the vessel wall).

The body may be a flexible catheter formed from any material suitable for intravascular use, such as, but not limited to, silicone, rubber, polyurethane, etc. The tip 34 may formed of any material suitable for intravascular use such as, silicone, rubber, polyurethane, or the like.

The catheter device further includes at least one vibrational pad 30. The vibrational pad may be any device configured to vibrate at a frequency and/or amplitude sufficient facilitate occlusive material removal. For example, the at least one vibrational pad 30 may include piezo-electric actuator, motors, or the like which may be traversed through the vasculature of a subject to a target location. More particularly, in some embodiments, the at least one vibrational pad 30 may include a piezoelectric transducer capable of vibrating at a frequency between 1 Hz and 800 MHz. However, it should be understood that the catheter device 10 may be configured to operate across an entire range of frequencies achievable by the particular vibrational pad selected for use in the catheter device. In these embodiments, oscillation between an upper bound and a lower bound of the frequency achievable by the at least one vibrational pad 30 may help ensure that the at least one vibrational pad 30 achieves a frequency which is sufficient to disintegrate and/or dislodge any occlusive material that contact the vibrational pad 30. It should be further understood that the upper bound and the lower bound of the frequency achievable by a particular vibrational pad 30 may vary based on the physical properties (e.g., length, thickness, weight, etc.) of the at least one vibrational pad 30.

Referring still to FIG. 1, the at least one vibrational pad 30 may be housed within the at least one lateral opening 28. In these embodiments, the at least one vibrational pad 30 may be formed of a metal material, such as stainless steel, titanium, or any other similarly rigid material capable of transmitting vibrational energy to the target location. Furthermore, in some embodiments, the at least one vibrational pad 30 may include a coating, such as a diamond coating, that may provide the at least one vibrational pad with an enhanced grit for removing occlusive material positioned with the target location.

In these embodiments, the at least one vibrational pad may further include a contact surface 31, which may be configured to contact plaque located at a target site during an occlusion removal process.

Referring now to FIGS. 1-3, the catheter device 10 may further include at least one actuator 32 coupled to the at least one vibrational pad 30, such that the actuator 32 may move the at least one vibrational pad in a transverse (e.g., perpendicular to the catheter device 10 in the +/−y-direction as depicted in the coordinate axes of FIGS. 1-3) direction. In embodiments, the at least one actuator 32 may be housed within the lumen 26, and may be used to actuate the at least one vibrational pad 30 between a first position and a second position. For example, the actuator 32 may include a slide lever, a gear train, a wheel pulley, or any other similar actuator that may be manually or electromechanically actuated between a first position and a second position.

In the first position, the at least one vibrational pad 30 may be retracted such that the contact surface 31 is relative to or flush with an external surface 21 of the body 20, while in the second position, the at least one vibrational pad 30 may radially protrude, such that the contact surface 31 extends laterally from the external surface 21 of the body 20 a distance further than when in the first position, as will be described in additional detail herein.

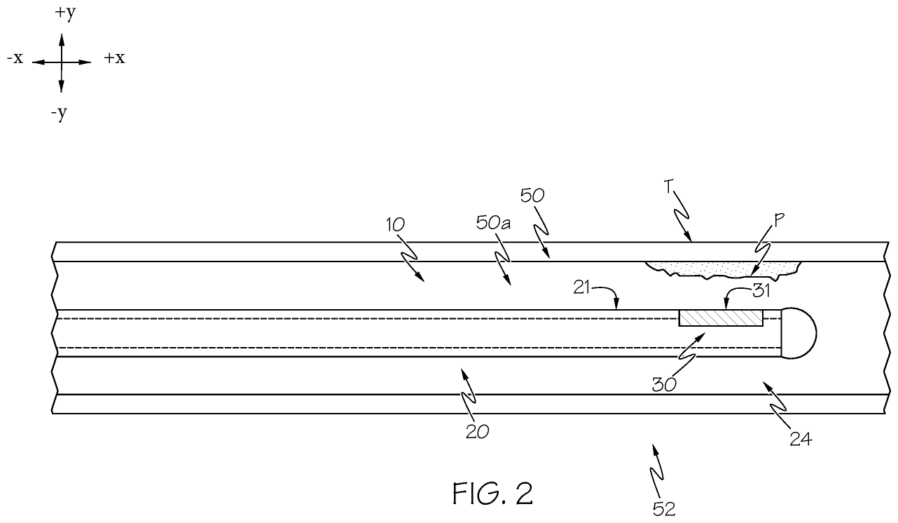

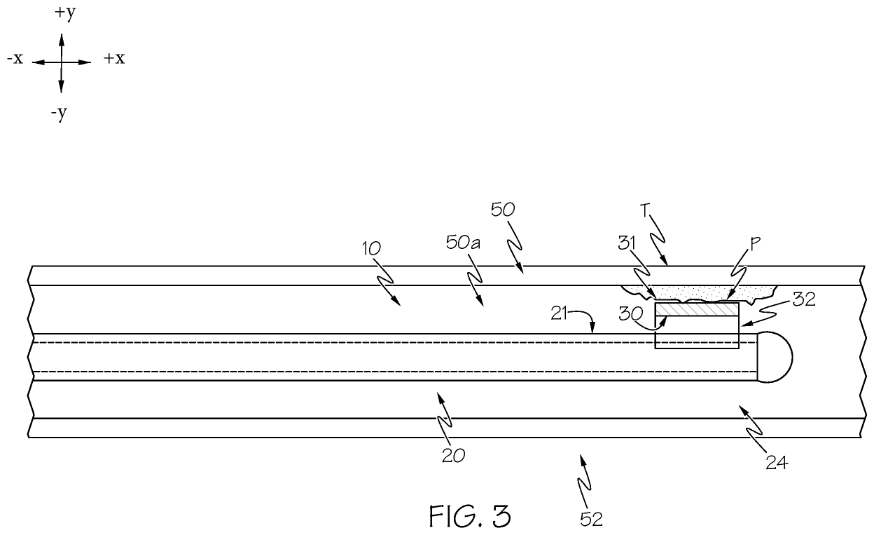

In some embodiments, the contact surface 31 of the at least one vibrational pad 30 may be a compliant surface, such as a sponge or foam (e.g., metal shape alloys, polymer, composite, etc.) or any other similarly compressible material, such that the contact surface 31 is capable of conforming to the arterial wall and any plaque located on the vessel wall 50. For example, as the actuator 32 extends the at least one vibrational pad 30 to the second position, the contact surface 31 may become compressed between the at least one vibrational pad and plaque located on the vessel wall 50, which may cause the contact surface 31 to conform to plaque positioned on the vessel wall 50. By conforming the contact surface 31 to the arterial wall and any plaque located one the arterial wall, the vibration of the at least one vibrational pad 30 may more effectively dislodge and/or disintegrate the plaque P. Although FIGS. 2 and 3 illustrate the contact surface 31 having a compliant surface, it should be understood that some embodiments may include a contact surface 31 having a non-compliant surface, such that the contact surface 31 does not conform to the vessel wall and any plaque positioned on the vessel wall.

The catheter device may further include a controller 100, which may be communicatively coupled to the at least one actuator 32 and the vibrational pad 30, such that the controller 100 is operable to control the actuation of the at least one actuator 32 and the amplitude and frequency of the vibration of the at least one vibrational pad 30. Although the embodiments illustrated herein depict the controller 100 being communicatively coupled to the at least one actuator 32 and the vibrational pad 30, it should be understood that, in some embodiments, the controller 100 may only be communicatively coupled to the at least one vibrational pad 30. For example, in some embodiments, the at least one actuator 32 may be manually driven, such that a user may manually control actuation of the at least one actuator 32. Although not depicted, in these embodiments, the at least one actuator 32 may include a handle, a lever, a push button, or any other similar mechanism that may be capable of affecting actuation of the at least one actuator 32. For example, in embodiments in which the at least one actuator includes a lever mechanism, the lever may be configured to rotate between a first position and a second position. As the lever rotates from the first position to the second position, rotation of the lever may cause the at least one pad 32 to translate in a transverse direction between the first position and the second position.

By controlling the actuation of the at least one actuator 32, a user may ensure that the contact surface 31 of the at least one vibrational pad 30 is in contact with the plaque located at a target site before energy is provided to the at least one vibrational pad 30. Furthermore, by controlling the amplitude and frequency of the vibration of the at least one vibrational pad 30, a user can ensure that a high enough frequency is used to effectively disintegrate and/or dislodge the plaque located at a target site without damaging the arterial wall. Furthermore, by controlling the amplitude and frequency of the at least one vibrational pad 30, a user may be able to effectively disintegrate and/or dislodge various types of plaque positioned at the target site. For example, the at least one vibrational pad 30 may be adjusted to treat luminal plaque, sub-intimal plaque, and/or medial plaque positioned at the target site. In these embodiments, it should be understood that sub-intimal plaque may be defined as plaque positioned within an intima of a vessel wall, while medial plaque may be defined as plaque positioned within a media of a vessel wall.

In these embodiments, a user may further control the vibrational direction of the at least one vibrational pad 30 in order to effectively disintegrate and/or dislodge the plaque located at the target site. For example, the at least one vibrational pad 30 may be controlled to vibrate in a longitudinal direction (e.g., in the +/−x-direction as depicted in the coordinate axis of FIG. 1) or in a lateral direction (e.g., in the +/−y-direction as depicted in the coordinate axis of FIG. 1) depending on the size and/or shape of the plaque positioned at the target site. In these embodiments, the vibrational direction (e.g., longitudinal and/or lateral) may be controlled via the orientation of the at least one vibrational pad. For example, in some embodiments, the at least one vibrational pad 30 may be a flat vibrational pad oriented along the longitudinal axis (e.g., along the +/−x-axis depicted in FIG. 1), such that, when the at least one vibrational pad 30 is activated, the vibrations caused by the at least one vibrational pad 30 oscillate along the longitudinal axis. In contrast, the at least one vibrational pad 30 may be rotated 90 degrees in order to allow for oscillation of the at least one vibrational pad in the lateral direction.

Turning now to FIGS. 2 and 3, the catheter device 10 is depicted in a lumen of a target site T. In these embodiments, the target site T may be a location within a vessel 52 where an occlusion removal process is performed, and may correspond to a location within the vessel where a buildup of plaque is positioned.

Referring still to FIGS. 2 and 3, the at least one vibrational pad 30 may be actuatable between the first position (FIG. 2) and the second position (FIG. 3), respectively. In the first position, the contact surface 31 of the vibrational pad 30 may lie substantially flush with an external surface 21 of the body 20. In these embodiments, the contact surface 31 of the at least one vibrational pad may be considered the surface of the pad which directly contacts the plaque P located at the target site T.

In some embodiments, the at least one vibrational pad 30 may be positioned in the first position before the catheter device 10 is initially inserted into a vessel. In these embodiments, the distal end 24 of the body 20 may be inserted into the vessel and advanced in a longitudinal (+x) direction until the at least one vibrational pad 30 is positioned at the target site T. With the contact surface 31 of the at least one vibrational pad 30 sufficiently flush with the external surface 21 of the body 20 of the catheter device 10, the catheter device 10 may navigate an area within a vessel wall 50 of the vessel 52. For example, the vessel wall 50 may include a sub-intimal layer and a medial layer and may further define a vessel lumen 50a through which the catheter device 10 traverses in the longitudinal direction. In these embodiments, the alignment of the at least one vibrational pad 30 with the external surface 21 of the body 20 may ensure that the catheter device 10 is able to traverse the vessel 52 in an obstructed manner, and may prevent reduce unwanted contacted with the vessel wall 50 during traversal of the catheter device 10 through the vessel 52.

Referring now to FIG. 3, the at least one vibrational pad 30 is illustrated in the second position. To alternate the at least one vibrational pad 30 from the first position to the second position, the controller 100 may activate the actuator 32 such that the at least one vibrational pad 30 moves in a transverse direction relative to the lumen 26 of the body 20 of the catheter device 10. The actuator 32 may continue to move the at least one vibrational pad 30 in the transverse direction until the contact surface 31 of the at least one vibrational pad 30 contacts the plaque P located at the target site T. In the embodiments described herein, a user may determine that the contact surface 31 of the at least one vibrational pad 30 has contacted plaque P located at the target site T using a fluoro-imaging process, or other similar process. For example, a contrast injection may be applied to the target site T prior to the catheter device 10 being inserted into the target site T, such that, when utilizing a fluoro-imaging process, a user is able to identify the position of the catheter device 10 and its various components in relation to the target site T and any plaque P located at the target site T.

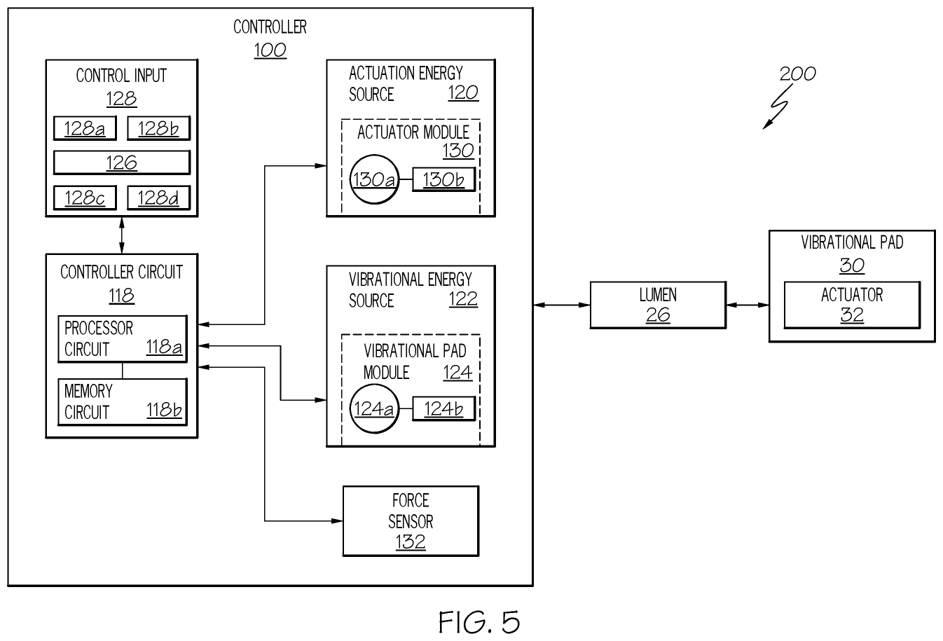

For example, FIG. 3 illustrates plaque P fixed to a surface of the vessel wall 50. In these embodiments, the catheter device 10 may traverse the vessel lumen 50a in the longitudinal direction until the at least one vibrational pad 30 is aligned with the plaque P. With the catheter device 10 positioned, the controller 100 may activate the actuator 32, which may in turn cause the at least one vibrational pad 30 to move in the transverse direction until the contact surface 31 of the vibrational pad 30 contacts the sub-intimal plaque P. The at least one vibrational pad 30 may then be activated by the controller 100 in order to dislodge the plaque P from the target site T. In some embodiments, the actuator 32 may be further configured to generate vibration of the at least one vibrational pad in order to dislodge the plaque P from the target site T. However, in other embodiments, the at least one vibrational pad 30 may be separately wired to the controller 100, such that the controller 100 may facilitate activation of the at least one vibrational pad 30 when the actuator 32 has moved the contact surface 31 of the vibrational pad 30 into contact with plaque P. Operation of the controller 100 will be described in additional detail herein with reference to FIG. 5.

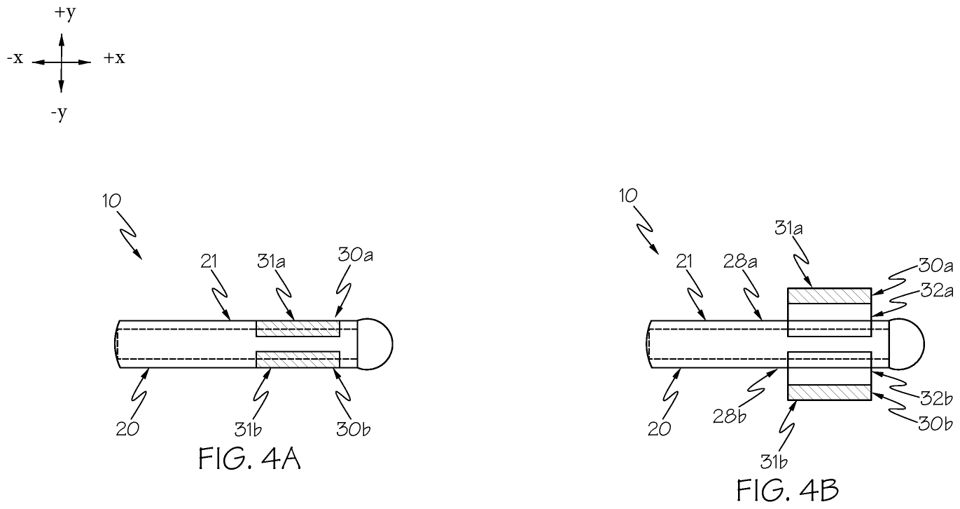

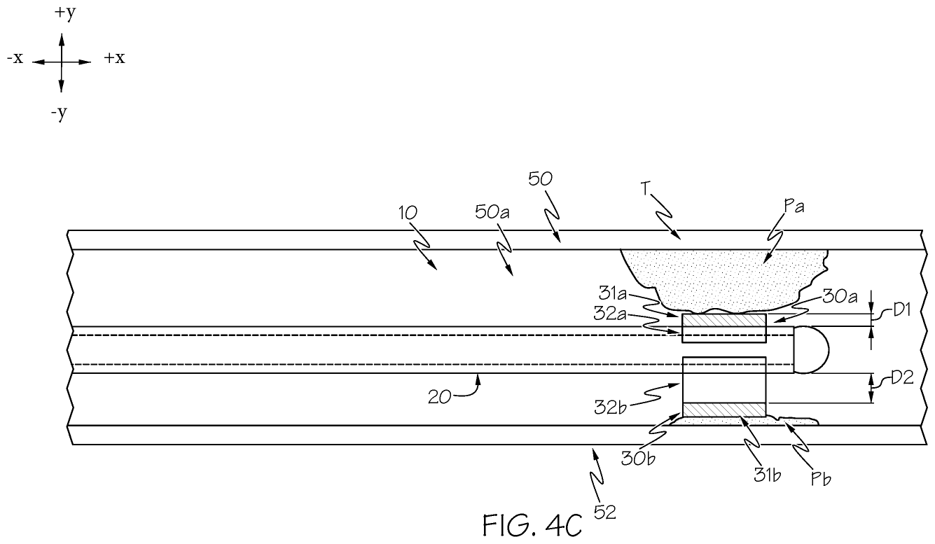

Referring now to FIGS. 4A and 4B, in some embodiments, the one or more vibrational pads 30 may include a plurality of vibrational pads, such as a first vibrational pad 30a and a second vibrational pad 30b, which may be positioned circumferentially about the external surface 21 of the body 20 of the catheter device 10. Although FIGS. 4A and 4B illustrate the catheter device 10 having two vibrational pads 30a, 30b, it should be understood that the catheter device 10 may include any number of vibrational pads which may be circumferentially arranged about the external surface 21 of the body 20 of the catheter device 10. For example, in some embodiments, the catheter device 10 may include three vibrational pads 30, four vibrational pads 30, etc.

As in the embodiments above, each of the vibrational pads 30a 30b may further include a contact surface 31. For example, as illustrated in FIGS. 4A and 4B, the first vibrational pad 30a may include a first contact surface 31a, and the second vibrational pad 30b may include a second contact surface 31b. In these embodiments, the first contact surface 31a and the second contact surface 31b may both be compliant surfaces, such that the first and second contact surfaces 31a, 31b may conform to the arterial wall and/or plaque located at the target site. However, in some embodiments, the first contact surface 31a and second contact surface 31b may both include non-compliant contact surfaces 31. In other embodiments still, the first contact surface 31a may be a compliant surface, while the second contact surface 31b may be a non-compliant surface, or vice versa.

Referring still to FIGS. 4A and 4B, the catheter device 10 may include at least one lateral opening 28 for each of the plurality of vibrational pads 30. For example, the catheter device 10 depicted in FIGS. 4A and 4B may include a first lateral opening 28a, in which the first vibrational pad 30a is housed, and a second lateral opening 28b, in which the second vibrational pad 30b is housed. Similarly, the catheter device 10 may include at least one actuator 32 for each of the plurality of vibrational pads 30. For example, the catheter device 10 may include a first actuator 32a coupled to the first vibrational pad 30a, and a second actuator 32b coupled to the second vibrational pad 30b. Although FIGS. 4A and 4B depict the catheter device 10 as having a first actuator 32a and a second actuator 32b, it should be noted that, in some embodiments, the catheter device 10 may include a single actuator 32 that may be used to actuate both of the first vibrational pad 30a and the second vibrational pad 30b between the first position and the second position.

Referring now to FIG. 4A, the catheter device 10 having the plurality of vibrational pads 30 is illustrated in the first position. In these embodiments, the first vibrational pad 30a and the second vibrational pad 30b may both be substantially flush with the external surface 21 of the body 20 in the first position. As discussed herein, the alignment of the first vibrational pad 30a and the second vibrational pad 30b with the external surface 21 of the body 20 may ensure that the catheter device 10 is able to traverse the vessel in an obstructed manner, and may help prevent excessive contact between the vessel wall 50 and the first and or second vibrational pad 30a, 30b while the catheter device 10 is being positioned.

Turning now to FIG. 4B, the first vibrational pad 30a and the second vibrational pad 30b are illustrated in the second position. In these embodiments, the controller 100 may be communicatively coupled to both the first actuator 32a and the second actuator 32b, such that the controller 100 may alternate the first and second vibrational pads 30a, 30b from the first position to the second position, and activate the first and second actuators 32a, 32b such that the first and second vibrational pads 30a, 30b move in a transverse direction. The first and second actuators 32a, 32b may continue to move the first and second vibrational pads 30a, 30b in the transverse direction until the contact surface 31 of each of the vibrational pads 30a, 30b contacts the plaque located at the target site.

In some embodiments, the controller 100 may be configured to activate the first actuator 32a and the second actuator 32b simultaneously, such that the first and second actuators 32a, 32b move the first and second vibrational pads 30a, 30b in unison. However, in other embodiments, it may be desirable for the controller 100 to be configured to activate the actuators 32a, 32b independently, such that the transverse movement of each of the vibrational pads 30a, 30b may be controlled individually.

As illustrated in FIG. 4C, the amount of plaque buildup on the vessel wall 50 may influence the amount of transverse actuation needed to bring the first contact surface 31a of the first vibrational pad 30 or the second contact surface 31b of the second vibrational pad 30b into contact with the plaque P and/or arterial wall. For example, the plaque buildup Pa on a first portion of the vessel wall 50 may be more substantial than the plaque buildup Pb on a second portion of the vessel wall 50. As a result, the first vibrational pad 30a may be actuated in the transverse direction a first distance D1 such that the first contact surface 31a contacts plaque buildup Pa, while the second vibrational pad 30b may be actuated in the transverse direction a second distance D2 such that the second contact surface 31b contacts plaque buildup Pb. For example, contact may be determined such as via fluoroscopy.

In such embodiments, the first actuator 32a and the second actuator 32b may be activated separately once the catheter device 10 is positioned at the target site T. For example, once the catheter device 10 is positioned, the controller 100 may activate the first actuator 32a, which may move the first vibrational pad 30a the distance DI such that the first contact surface 31a contacts the plaque buildup Pa on the vessel wall 50. With the first vibrational pad 30a contacting the plaque buildup Pa, the controller 100 may then activate the second actuator 32b, which may move the second vibrational pad 30b the distance D2 such that the second contact surface 31b contacts the plaque buildup Pb on the vessel wall 50.

Referring still to FIG. 4C, the controller 100 may be configured to activate the plurality of vibrational pads 30 once the vibrational pads 30 have been actuated to contact plaque at the target site. In some embodiments, the controller 100 may be configured to activate each of the plurality of vibrational pads 30 simultaneously, such that each of the vibrational pads 30 vibrate in unison. In these embodiments, each of the plurality of vibrational pads 30 may vibrate at the same frequency and amplitude in order to disintegrate and/or dislodge plaque on the vessel wall 50 at the target site T.

In other embodiments, the controller 100 may be further configured to activate the plurality of vibrational pads 30 individually. For example, the density of the plaque deposited on the vessel wall 50 may influence the vibrational frequency and amplitude required to disintegrate and/or dislodge the plaque. As illustrated in FIG. 4C, the plaque buildup Pa located on the vessel wall 50 may be more substantial than the plaque buildup Pb located on the vessel wall 50. As a result, the first vibrational pad 30a may be vibrated at a higher frequency and amplitude to dislodge and/or disintegrate the plaque Pa on the vessel wall 50 Referring again to FIG. 4C, it should be noted that vibration of at least one of the plurality of vibrational pads 30 may cause the catheter device 10 to move in the transverse direction between through the vessel lumen 50a. Thus, vibration of one of the plurality of vibrational pads 30 may cause the contact surface 31 of another of the plurality of vibrational pads 30 to lose contact with the plaque and or arterial wall. For example, when the controller 100 activates the first vibrational pad 30a, the vibration of the vibrational pad 30a may cause the catheter device 10 to move in the transverse direction. As vibration from the vibrational pad 30a causes the plaque Pa located on the vessel wall 50 to become dislodged and/or disintegrated, the catheter device may move in the transverse direction (+y) towards the vessel wall 50. In these embodiments, the transverse (+y) movement of the catheter device 10 may cause the second contact surface 31b of the second vibrational pad 30b to lose contact with the plaque Pb located on the vessel wall 50. Thus, the second vibrational pad 30b may be repositioned such that the second contact surface 31b reengages the plaque Pb before the second vibrational pad 30b is activated, such positioning may be determined via fluoroscopy, for example.

In some embodiments, it may be beneficial to activate the first and second vibrational pads 30a, 30b individually in order to avoid exerting excessive vibrational force on the vessel. For example, depending on the vibrational frequency and amplitude required to dislodge plaque located on the vessel wall 50, the combined vibrational forces of the first vibrational pad 30a and the second vibrational pad 30b may exceed a predetermined threshold. However, by activating a single vibrational pad 30 at a time, the total vibrational force acting on the vessel may be reduced, such that the plaque deposited on the first vessel wall may be dislodged and/or disintegrated without excessive vibrational force being applied to the vessel.

Referring now to FIG. 5, a schematic view of the controller 100 for operating the catheter device 10 is illustrated. In these embodiments, the controller 100 and the catheter device 10 may form an atherectomy system 200, which may be utilized to perform atherectomy procedures, as will be described in additional detail herein. In some embodiments, the controller 100 may be configured to actuate the at least one vibrational pad 30 between the first position and the second position, and adjust the vibrational frequency and amplitude of the at least one vibrational pad 30. The controller 100 may include a controller circuit 118, a vibrational energy source 122, an actuation energy source 120, a force sensor 132, and a battery 126. The controller 100 may further include a control input 128, such as a computer, which may allow a user to operate the controller 100. In these embodiments, the control input 128 may be configured to provide control of the controller 100 via a software program, or may be manually controlled via a user manipulating a user interface thereof.

Referring still to FIG. 5, the control input 128 may include control buttons and visual/aural indicators, such as a display and/or speakers, with the control buttons providing user control over various functions of the controller 100, and with the visual/aural indicators providing visual/aural feedback of the status of one or more conditions and/or positions of components of the controller 100. For example, the display and/or speakers may provide an audible tone that indicates to a user that the catheter device 10 is positioned within the vessel, or that the at least one vibrational pad 30 is engaged with plaque positioned within the vessel. The control buttons may include one or more buttons for actuating the at least one vibrational pad between the first and second position 128a, 128b and one or more buttons and/or knobs 128c, 128d for adjusting the frequency and amplitude of the vibration of the at least one vibrational pad 30.

The controller circuit 118 is electrically and communicatively coupled to the actuation energy source 120, the vibrational energy source 122, the force sensor 132, and the control input 128, such as by one or more wires or circuit traces. The controller circuit 118 may be assembled on an electrical circuit and may include, for example, a processor circuit 118a and a memory circuit 118b.

Processor circuit 118a has one or more programmable microprocessors and associated circuitry, such as an input/output interface, buffers, memory, etc. The memory circuit 118b is communicatively coupled to processor circuit 118a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Controller circuit 18 may be formed as one or more Application Specific Integrated Circuits (ASIC).

The controller circuit 118 is configured via software and/or firmware residing in memory circuit 118b to execute program instructions to perform functions associated with the actuation of the at least one vibrational pad 30 between the first and second positions and/or frequency and amplitude of vibration of the at least one vibrational pad 30.

The actuation energy source 120 may include, for example, an actuator module 130. In these embodiments, the actuation energy source 120 may include an actuator module 130 for each of the vibrational pads 30 on the catheter device. For example, FIG. 5 illustrates a controller 100 for a catheter device having only a single vibrational pad 30. Thus, the actuation energy source 120 includes one actuator module 130. However, in embodiments in which the catheter device 10 includes a plurality of vibrational pads 30, the actuation energy source 120 may include a plurality of actuator modules 130. For example, a controller 100 configured to operate the catheter device of FIGS. 4A-4C may include a first actuator module and a second actuator module, which may be configured to actuate the first vibrational pad 30a and the second vibrational pad 30b, respectively.

Each of the actuator modules 130 may be electrically and controllably coupled to controller circuit 118. As provided herein, the actuator module 130 may be electrically coupled to the controller circuit 118 by way of electrical wiring or any other suitable electrical connections, such that user inputs on the control input 128 may be relayed to the controller circuit 118 and used to control the power delivered by the actuator module 130 to the at least one actuator 32. In these embodiments, the actuator module 130 may include a power supply 130a, such as an electric motor, to which an electric lead 130b is attached. In embodiments in which multiple actuator modules 130 are present, each actuator module may include a power source and an electric lead.

Referring still to FIG. 5, vibrational energy source 122 may include a vibrational pad module 124. The vibrational pad module may include, for example, a power supply 124a, such as an electric motor, that provides an activation current to the at least one vibrational pad 30 via an electric lead 124b. Although the vibrational pad module 124 and the actuator module 130 are depicted as having separate power supplies 124a, 130a, respectively, it should be further understood that, in some embodiments, each of the components of the controller 100 may be powered via a single power supply or be connected to a single power source.

In these embodiments, the vibrational energy source 122 may include a vibrational pad module 124 for each of the vibrational pads 30 on the catheter device. As previously discussed, FIG. 5 illustrates a controller 100 for a catheter device having only a single vibrational pad 30. Thus, the vibrational energy source 122 includes one vibrational pad module 124. However, in embodiments in which the catheter device 10 includes a plurality of vibrational pads 30, the vibrational energy source 122 may include a plurality of vibrational pad modules 124. For example, a controller 100 configured to operate the catheter device of FIGS. 4A-4C may include a first vibrational pad module and a second vibrational pad module, which may be configured to supply a first vibrational current to the first vibrational pad 30a and a second vibrational current to the second vibrational pad 30b. It should be understood that each vibrational energy source 122 may be electrically and controllably coupled to controller circuit 118.

In the embodiments described herein, the electrical leads 124b, 130b may extend from the controller 100 through the lumen 26 of the catheter device 10, such that the controller 100 is able to control the actuation and vibration of the at least one vibrational pad 30, respectively. Separate electrical leads may be provided for separate actuators where independent actuation of the actuators is desired. For example, once the catheter device 10 is positioned within the vessel, the actuation energy source 120 may be operated to cause the at least one actuator 32 to move the at least one vibrational pad 30 in the transverse direction from the first position to the second position, such that the contact surface 31 of the at least one vibrational pad 30 engages plaque deposited on and within the arterial wall. Once the vibrational pad 30 is in the second position, the vibrational energy source 122 may be operated to supply current to the at least one vibrational pad 30, causing the at least one vibrational pad 30 to vibrate. As the vibrational pad 30 vibrates, the force sensor 132 may output force feedback to the controller 100 to allow the controller 100 to monitor the vibrational force applied by the at least one vibrational pad 30 on the vessel wall. For example, in some embodiments, the force sensor 132 may determine the vibrational force applied by the at least one vibrational pad 30 on the vessel wall by monitoring an electrical current supplied to the vibrational pad 30. In these embodiments, it should be appreciated that a greater electrical current supplied to the vibrational pad 30 may correspond to a greater vibrational force, and vice versa.

In other embodiments, a user may utilize a fluoro-imaging process, as has been described herein, to determine when the plaque P has been dislodged and/or disintegrated at the target site T. Once the plaque deposited on the arterial wall has been dislodged and/or disintegrated, the controller circuit 118 may deactivate the vibrational energy source 122 in order to stop vibration of the at least one vibrational pad 30. The controller circuit may then reactivate the actuation energy source 120 such that the actuator 32 may move the vibrational pad 30 from the second position to the first position, at which point the catheter device 10 may be safely removed from the vessel.

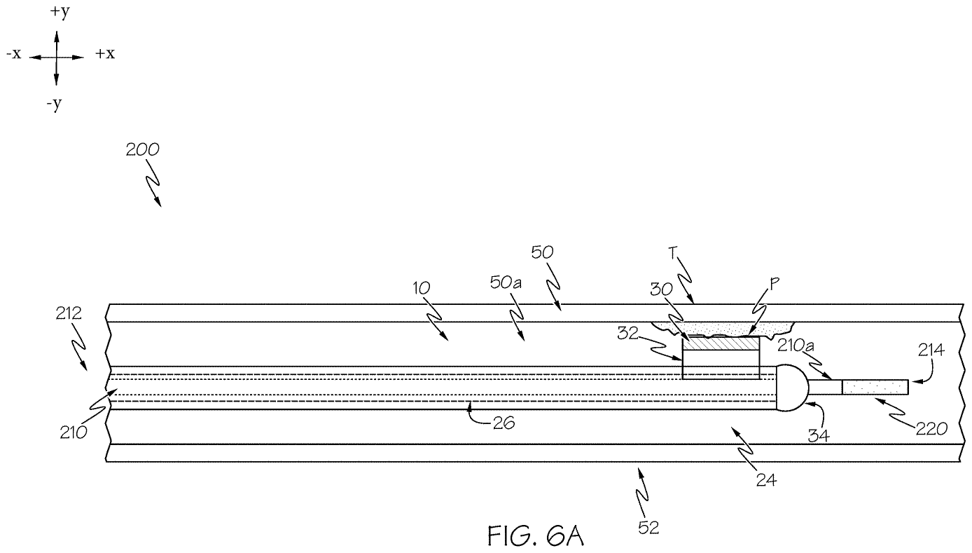

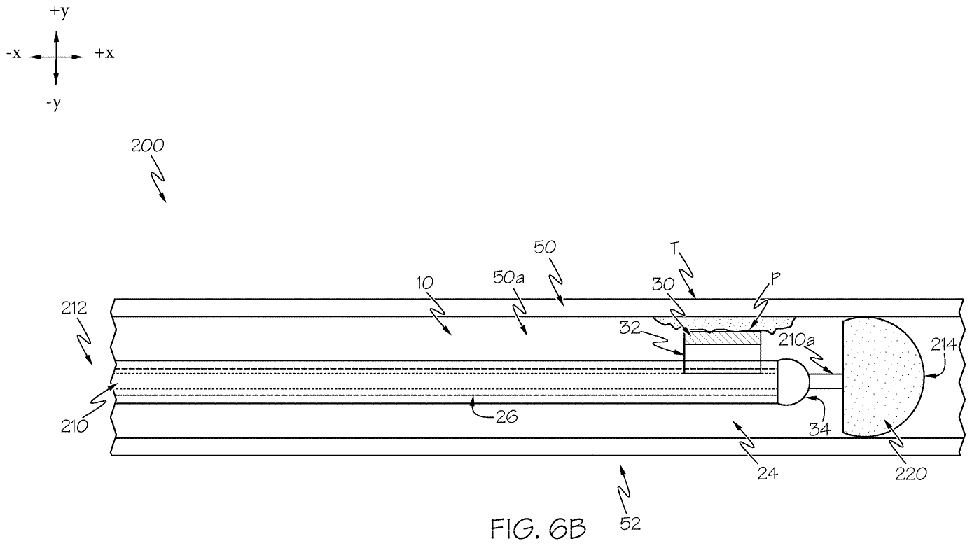

Turning now to FIGS. 6A and 6B, an atherectomy system 200 is disclosed. The atherectomy system 200 may include the catheter device 10, the controller 100 and may further include a filter 220. A filter tube 210 having a first end 212 and a second end 214 may be used to deploy the filter 220, with the filter 220 being coupled to the second end 214 of the filter tube 210. In such embodiments, the filter tube 210 may be inserted through the lumen 26 of the catheter device 10 and advanced in the longitudinal direction (+x) such that the filter 220 extends from the tip 34 of the body 20 and is positioned distal to the distal end 24 of the body 20 of the catheter device 10.

As further illustrated in FIGS. 6A and 6B, the filter 220 may be moved between a retracted position and an expanded position. In the retracted position, the filter 220 may completely surround the second end 214 of the filter tube 210, such that the filter 220 lies substantially flush with an outer surface 210a of the filter tube 210, as is shown in FIG. 6A. In these embodiments, the filter 220 may be compressed to the retracted position before being inserted into the catheter device 10, such that the filter 220 and filter tube 210 may pass through the lumen 26 of the body 20 of the catheter device 10 without interfering with the at least one vibrational pad 30 or at least one actuator 32.

Once the filter 220 is positioned, the filter may be moved to the expanded position, as is illustrated in FIG. 6B. In the expanded position, the filter 220 may extend across the vessel lumen 50a such that the filter 220 contacts the vessel wall 50. In these embodiments, the filter 220 may be expanded by providing an expansion medium, such as fluid, through the filter tube 210. In other embodiments, the second end 214 of the filter tube 210 may further include a retaining element, such as a clasp, clamp, pin, or the like, which may hold the filter 220 in the retracted position. In these embodiments, the filter 220 may be moved to the expanded position by releasing the retaining element. For example, the filter tube 210 may include a release mechanism which may be manually and/or electromechanically engaged to cause the retaining element to release the filter 220. Furthermore, in some embodiments, the filter 220 may be self-expandable, such that the filter 220 moves to the expanded position when positioned at the target site T. It should be understood that any filter having a retracted position and configured to be expanded to an expanded position to extend across the vessel lumen may be used. For example, in the embodiments described herein, the filter 220 may be formed of nitinol, nickel, titanium, or any other similar material that may allow the filter 220 to be compatible with a guidewire.

Referring still to FIG. 6B, when the catheter device 10 has disintegrated and/or dislodged the plaque positioned P at the target site T, the filter 220 may collect and remove the dislodged plaque P as the atherectomy system 200 is removed from the target site T. For example, once the plaque P illustrated in FIG. 6B is dislodged, the filter 220 may move in the longitudinal direction (−x) towards the dislodged plaque as the atherectomy system 200 is withdrawn from the vessel. Because the filter 220 may extend across the vessel lumen 50a to contact the vessel wall 50, the filter 220 may collect any dislodged plaque P as the filter is withdrawn in the longitudinal (−x) direction. By utilizing the filter 220, a user may ensure that all dislodged plaque P is removed from the target site T, thereby minimizing the risk of the dislodged plaque P traversing the vessel to another location within the patient.

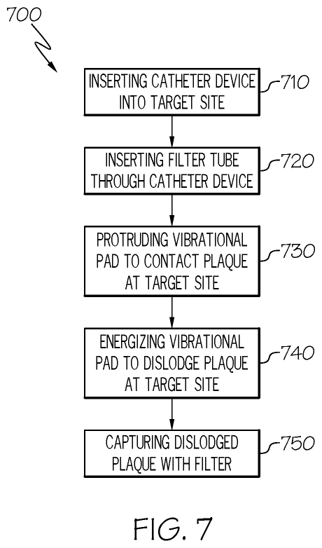

Turning now to FIG. 7, an illustrative flow diagram of a method of removing plaque from a target site 700 is shown. In these embodiments, the method may first involve inserting a distal end 24 of the catheter device 10 into a target site T, as shown at block 710. In some embodiments, this step may further involve positioning the distal end 24 of the catheter device 10 such that the at least one vibrational pad 30 is aligned with a region of plaque P at the target site T.

With the catheter device 10 positioned, the method may move to block 720, which may involve inserting filter tube 210 through the proximal end 22 of the body 20 and advancing the filter tube 210 in the longitudinal direction until the second end 214 of the filter tube 210 extends beyond the tip 34 of the body 20. In these embodiments, the method step illustrated at block 720 may further involve deploying the filter 220 positioned on the second end 214 of the filter tube 210 to the expanded position, such that the filter 220 extends across the vessel wall 50.

With the filter 220 deployed, the method may move to block 730, which may involve protruding the at least one vibrational pad 30 from the distal end 24 of the catheter device 10 such that the at least one vibrational pad 30 contacts plaque P located at the target site T. In these embodiments, the at least one actuator 32 may be activated by the controller 100 to actuate the at least one vibrational pad 30 in the transverse direction, until the contact surface 31 of the at least one vibrational pad 30 contacts the plaque P, as has been described herein. In embodiments in which the catheter device 10 includes a plurality of vibrational pads 30, the method step illustrated at block 730 may involve actuating each of the plurality of vibrational pads, such as first vibrational pad 30a and second vibrational pad 30b, until the contact surfaces 31a, 31b of each vibrational pad 30 are in contact with plaque P located at the target site T.

The method may then move to block 740, which may involve energizing the at least one vibrational pad 30 at a frequency and amplitude sufficient to dislodge plaque P located at the target site T. In embodiments in which the catheter device 10 includes the plurality of vibrational pads 30, the method step of block 740 may include energizing the plurality of vibrational pads 30 simultaneously, or energizing the plurality of vibrational pads individually, as has been described herein. Furthermore, in embodiments in which the catheter device 10 includes the plurality of vibrational pads 30 and each of the vibrational pads 30 are energized individually, the catheter device 10 may be repositioned after the first vibrational pad 30a is energized in order to ensure that the second vibrational pad 30b remains in contact with plaque P located at the target site T.

When the plaque P positioned at the target site T has been disintegrated and/or dislodged, the method may move to block 750, which may involve capturing the dislodged and/or disintegrated plaque P with the filter 220. In these embodiments, as the catheter device 10 and filter tube 210 are being removed from the target site T, the dislodged plaque may be captured by the filter 220 and removed from the target site T along with the catheter device 10 and filter tube 210.

Embodiments may be further described with references to the following numbered clauses:
1. A catheter device comprising: a body extending between a proximal portion and a distal portion and defining a lumen and at least one lateral opening in communication with the lumen at the distal portion; and at least one vibrational pad positionable within the lumen and configured to adjustably protrude radially through the at least one lateral opening, wherein the at least one vibrational pad is configured to be energized to break apart sub-intimal or medial plaque.
2. The catheter device of clause 1, wherein the at least one vibrational pad comprises a contact surface configured to deliver vibrational energy to a target site, wherein the at least one vibrational pad is configured to move from a first position in which the contact surface is substantially flush with an external surface of the body to a second position in which the contact surface is spaced radially outward from the external surface of the body.
3. The catheter device of clauses 2 or 3, further comprising a least one actuator coupled to the at least one vibrational pad and operable to move the vibrational pad from the first position to the second position.
4. The catheter device of any of clauses 1-3, further comprising a controller communicatively coupled to the at least one vibrational pad and operable to control vibration of the at least one vibrational pad.
5. The catheter device of any of clauses 1-4, comprising a piezo actuator, wherein the at least one vibrational pad is configured to be energized by the piezo actuator.
6. The catheter device of any of clauses 1-5, further comprising a controller coupled to the proximal portion of the body, wherein the controller is communicatively coupled to the at least one vibrational pad and operable to control vibration of the at least one vibrational pad.
7. The catheter device of any of clauses 1-6, wherein the at least one vibrational pad comprises a plurality of vibrational pads.
8. The catheter device of any of clauses 1-7, wherein the plurality of vibrational pads are arranged circumferentially about the body.
9. The catheter device of any of clauses 1-8, wherein the at least one vibrational pad further includes a compliant contact surface.
10. The catheter device of any of clauses 1-9, further comprising a controller communicatively coupled to the at least one vibrational pad and operable to control a vibration frequency and amplitude of the at least one vibrational pad.
11. The catheter device of any of clauses 1-10, wherein the vibrational frequency and amplitude are sufficient to break apart sub-intimal plaque from a target site.
12. The catheter device of any of clauses 1-11, wherein the vibrational frequency and amplitude are sufficient to break apart medial plaque from a target site.
13. An atherectomy system comprising: a catheter comprising: a body extending between a proximal portion and a distal portion and defining a lumen and at least one lateral opening in communication with the lumen at the distal portion; at least one vibrational pad positionable within the lumen and configured to adjustably protrude radially through the at least one lateral opening, wherein the at least one vibrational pad is configured to be energized at a frequency and amplitude sufficient to break apart sub-intimal or medial plaque; and a filter configured to be positioned distal to the distal portion of the catheter, the filter having a retracted position and an expanded position in which the filter is configured to extend across the lumen.
14. The atherectomy system of clause 13, comprising a piezo actuator wherein the at least one vibrational pad is configured to be energized by the piezo actuator.
15. The atherectomy system of any of clauses 13-14, wherein the distal portion of the catheter further comprises an atraumatic tip, preferably a silicon tip.
16. The atherectomy system of any of clauses 13-15, wherein the at least one vibrational pad is configured to be energized at a frequency and amplitude sufficient to break apart sub-intimal plaque from a target site.
17. The atherectomy system of any of clauses 16, wherein the at least one vibrational pad is configured to be energized at a frequency and amplitude sufficient to break apart medial plaque from a target site.
18. A method of removing plaque from a target site comprising: introducing a distal portion of a catheter having at least one vibrational pad to the target site; protruding the at least one vibrational pad from the distal portion of the catheter such that the at least one vibrational pad contacts plaque located at the target site; energizing the at least one vibrational pad such that vibration of the at least one vibrational pad dislodges plaque located at the target site; and capturing, using a filter device positioned in the distal portion of the catheter, the plaque dislodged from the target site.
19. The method of clause 18, wherein the at least one vibrational pad is a piezo actuator.
20. The method of any of clauses 18 or 19, wherein the at least one vibrational pad comprises a plurality of vibrational pads.

As should be appreciated in view of the foregoing, a catheter device is described herein. The catheter device may include a body extending between a proximal end and a distal end, such that the body defines a lumen and has at least one lateral opening in communication with the lumen at the distal end. At least one vibrational pad is positioned within the lumen and adjustably protrudes radially through the at least one lateral opening. The at least one vibrational pad is energized at a frequency and amplitude sufficient to break apart sub-intimal or medial plaque.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed clauses. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter device comprising:
a body extending between a proximal portion and a distal portion and defining a lumen and at least one lateral opening in communication with the lumen at the distal portion; and
at least one vibrational pad positionable within the lumen and configured to adjustably protrude radially through the at least one lateral opening, wherein the at least one vibrational pad is configured to be energized to break apart sub-intimal or medial plaque.

2. The catheter device of claim 1, wherein the at least one vibrational pad comprises a contact surface configured to deliver vibrational energy to a target site, wherein the at least one vibrational pad is configured to move from a first position in which the contact surface is substantially flush with an external surface of the body to a second position in which the contact surface is spaced radially outward from the external surface of the body.

3. The catheter device of claim 2, further comprising a least one actuator coupled to the at least one vibrational pad and operable to move the vibrational pad from the first position to the second position.

4. The catheter device of claim 1, further comprising a controller communicatively coupled to the at least one vibrational pad and operable to control vibration of the at least one vibrational pad.

5. The catheter device of claim 1, comprising a piezo actuator, wherein the at least one vibrational pad is configured to be energized by the piezo actuator.

6. The catheter device of claim 1, further comprising a controller coupled to the proximal portion of the body, wherein the controller is communicatively coupled to the at least one vibrational pad and operable to control vibration of the at least one vibrational pad.

7. The catheter device of claim 1, wherein the at least one vibrational pad comprises a plurality of vibrational pads.

8. The catheter device of claim 7, wherein the plurality of vibrational pads are arranged circumferentially about the body.

9. The catheter device of claim 1, wherein the at least one vibrational pad further includes a compliant contact surface.

10. The catheter device of claim 1, further comprising a controller communicatively coupled to the at least one vibrational pad and operable to control a vibration frequency and amplitude of the at least one vibrational pad.

11. The catheter device of claim 10, wherein the vibrational frequency and the amplitude are sufficient to break apart sub-intimal plaque from the target site.

12. The catheter device of claim 10, wherein the vibrational frequency and amplitude are sufficient to break apart medial plaque from the target site.

13. An atherectomy system comprising:
a catheter comprising:
a body extending between a proximal portion and a distal portion and defining a lumen and at least one lateral opening in communication with the lumen at the distal portion;
at least one vibrational pad positionable within the lumen and configured to adjustably protrude radially through the at least one lateral opening, wherein the at least one vibrational pad is configured to be energized at a frequency and amplitude sufficient to break apart sub-intimal or medial plaque; and
a filter configured to be positioned distal to the distal end of the catheter, the filter having a retracted position and an expanded position in which the filter is configured to extend across the lumen.

14. The atherectomy system of claim 13, comprising a piezo actuator, wherein the at least one vibrational pad is configured to be energized by the piezo actuator.

15. The atherectomy system of claim 13, wherein the distal end of the catheter further comprises an atraumatic tip, preferably a silicon tip.

16. The atherectomy system of claim 13, wherein the at least one vibrational pad is configured to be energized at a frequency and amplitude sufficient to break apart sub-intimal plaque from a target site.

17. The atherectomy system of claim 13, wherein the at least one vibrational pad is configured to be energized at a frequency and amplitude sufficient to break apart medial plaque from a target site.

18. A method of removing plaque from a target site comprising:
introducing a distal end of a catheter having at least one vibrational pad to the target site;
protruding the at least one vibrational pad from the distal end of the catheter such that the at least one vibrational pad contacts plaque located at the target site;
energizing the at least one vibrational pad such that vibration of the at least one vibrational pad dislodges plaque located at the target site; and
capturing, using a filter device positioned in the distal end of the catheter, the plaque dislodged from the target site.

19. The method of claim 18, wherein the at least one vibrational pad is a piezo actuator.

20. The method of claim 18, wherein the at least one vibrational pad comprises a plurality of vibrational pads.
